SEQUENCE LISTING (1 sequence)

SEQ ID NO: 1 - protein, 15 aa, Artificial
  Synthetic peptide binding to A beta oligomer
KRKKSRYKSWSVYVG

(19) United States
(12) Patent Application Publication
(10) Pub. No.: US 2025/0340593 A1
(43) Pub. Date: Nov. 6, 2025

(54) METHOD AND COMPOSITION FOR TREATING NEURODEGENERATIVE DISORDER

(71) Applicants: INSTITUT NATIONAL DE LA SANTÉ ET DE LA RECHERCHE MÉDICALE, Paris (FR); CENTRE NATIONAL DE LA RECHERCHE SCIENFITIQUE (CNRS), Paris (FR); UNIVERSITÉ CLAUDE BERNARD - LYON 1, Villeurbanne (FR); UNIVERSITE JEAN MONNET SAINT ETIENNE, Saint Etienne (FR)

(72) Inventors: Claire MEISSIREL, Lyon (FR); Pauline BOUVET, Lyon (FR); Laurent MARTIN, Tucson, AZ (US); Paul SALIN, Lyon (FR)

(21) Appl. No.: 18/860,909

(22) PCT Filed: May 5, 2022
(86) PCT No.: PCT/IB2022/000247

§ 371 Date: Oct. 28, 2024

(51) Int. Cl. C07K 7/08; A61K 38/00; A61P 25/28

(52) U.S. Cl. CPC C07K 7/08; A61P 25/28; A61K 38/00

(57) ABSTRACT

Inventors have shown evidence of VEGF accumulation in extracellular Aβ plaques in the post-mortem brain of patients with Alzheimer's disease (AD) and of the APP/PS1 mouse model of AD. They identified specific binding domains involved in the direct interaction between A0o and VEGF and engineered a peptide that blocks this interaction. The designed peptide binds to Aβ oligomers with high affinity and inhibits the process of Aβ self-aggregation, leading to the blockade of fibrillar aggregation. Furthermore, the peptide prevents soluble Aβ-derived toxins to target synapses in hippocampal neuron cultures and restores long-term potentiation in the hippocampus of the APP/PS1 mouse model of Alzheimer's disease. Thus, these findings have broad implications for preventing and treating diseases with Aβ neurotoxicity such as Alzheimer's disease. Accordingly, the invention relates to a peptide comprising the amino acid sequence KRKKSRYKSWSVYVG (SEQ ID NO: 1).

### FIELD OF THE INVENTION

The invention is in the field of neurology, more particularly the invention relates to method and composition for treating neurodegenerative disorder such as Alzheimer disease.

### BACKGROUND OF THE INVENTION

Alzheimer disease (AD), the most common form of dementia among elderly people, causes a progressive decline in memory and cognitive abilities (Dubois et al., 2014). Compelling evidence now indicate that β-amyloid peptide (Aβ) is a key player in AD with soluble forms of Aβ rather than insoluble fibrils correlating with the severity of cognitive symptoms, in link with synapse loss (Lue et al., 1999; McLean et al., 1999). Aβ oligomers (Aβo) are considered the most toxic species because they induce neuron and synapse damage, whether they are derived from patients with AD, from mouse or cellular models of the disease, or from synthetic preparations (Lambert et al., 1998; Walsh et al., 2002; Lesne et al., 2006). Furthermore, membrane-bound Aβo have revealed their ability to target synapses in living neurons (Lacor et al., 2004, 2007), by progressively concentrating into immobile clusters (Renner et al., 2010). In postmortem human brain, Aβo accumulate at postsynaptic sites as demonstrated by the combination of high-resolution threedimensional (3D) imaging and biochemical fractionation approaches (Koffie et al., 2012). In addition, they have also been shown to concentrate in presynaptic terminals in the APP/PSI mouse model of AD using super-resolution imaging and electron microscopy (Pickett et al., 2016). Their accumulation at both sides of the synapse causes major impairments in synapse function as demonstrated in the hippocampus using synthetic preparations or oligomeric preparations derived from human Aβ-overexpressing cells or from postmortem human AD brains. Both presynaptic and postsynaptic mechanisms have been involved in Aβo toxicity (Ting et al., 2007) and eventually result in the suppression of neurotransmitter release (He et al., 2019) and in the aberrant clustering and/or activation of postsynaptic glutamate receptors. Notably, extensive studies have demonstrated that Aβo from various sources were sufficient to strongly inhibit long-term potentiation (LTP) (Lambert et al., 1998; Walsh et al., 2002; Shankar et al., 2008) and facilitate long-term depression (LTD) in wild-type mice (Shankar et al., 2008, Li et al. 2009).

As the best neural correlate of memory impairments in AD is the shrinkage of the hippocampal region, in link with synapse loss (Chen et al. 2021), neurotrophic factors may counteract this loss and slow the progression of the disease. The vascular endothelial gowth factor (VEGF), best known for its angiognic role, has been shown to regulate key processes in the adult brain and in particular to promote hippocampal synaptic plasticity and memory consolidation (Cao et al., 2004; Kim et al., 2008; Licht et al., 2011; De Rossi et al., 2016). Importantly, VEGF gain of function in the rodent hippocampus substantially improves associative memory performances independently from its action on the vascular network, and even after a transient VEGF exposure (Licht et al., 2011). A recent study in trangenice mice models further revealed that the facilitating effect of VEGF on hippocampal synaptic plasticity and memory consolidation is due to its direct action on VEGFR2 expressing hippocampal neurons (De Rossi et al., 2016). In pathological conditions, the inventors highlighted a vicious cycle leading to the dysregulation of VEGF in the brain of AD patients and of the APP/PSI mouse model of the disease (Martin et al. 2021). Indeed, they showed that VEGF positive effect may grow weaker with time as Aβ plaque burden increases, due to its co-localization and potential sequestration in and around Aβ plaques. However, proper neuronal function requires VEGF signaling because its disruption has a negative impact on synaptic plasticity and memory consolidation. Along these lines the inventors revealed that an increase in VEGF supply in AD models can rescue the function of synapses confronted to Aβo toxicity, with the maintenance of their glutamate receptor content, the restoration of synaptic plasticity and the reduction in synapse loss (Martin et al. 2021).

Interestingly, the inventors further showed that VEGF which improves memory consolidation in mice and inhibits Aβo toxic action on synapses is selectively targeted by Aβo (Martin et al. 2021). This direct interaction between VEGF and Aβo opens new possibilities for treating subjects suffering from AD.

### SUMMARY OF THE INVENTION

The invention relates to a method for treating a subject suffering from a neurodegenerative disorder comprising a step of administering said subject with a therapeutically effective amount of an inhibitor of the interaction between amyloid-beta oligomers (Aβo) and vascular endothelial growth factor (VEGF). In particular, the invention is defined by the claims.

### DETAILED DESCRIPTION OF THE INVENTION

Inventors have shown the immunohistochemical evidence of VEGF accumulation in extracellular Aβ plaques in the post-mortem brain of AD patients and of the APP/PSI mouse model of AD. Based on this potential interaction between Aβ and VEGF, they further identified specific binding domains of the VEGF protein which are selectively targeted by Aβo. Next, they designed a new peptide tool that mimic one interaction domain in particular between Aβo and VEGF.

Importantly, inventors designed a peptide that binds to Aβ oligomers with high affinity and inhibits the process of Aβ self-aggregation, leading to the blockade of fibrillar aggregation. This peptide prevents soluble Aβ-derived toxins to target synapses in hippocampal neuron cultures. Furthermore, it rescues long-term potentiation (LTP) in the APP/PS1 mouse model of Alzheimer's disease. Thus, these findings have broad implications for preventing and treating diseases with Aβ neurotoxicity such as Alzheimer's disease.

### Peptide of the Invention

Accordingly, in a first aspect, the invention relates to a peptide comprising the amino acid sequence KRKKSRYKSWSVYVG (SEQ ID NO: 1).

In one embodiment, the peptide of the invention consists in the amino acid sequence as set forth in SEQ ID NO:1 comprising at least 75%, preferably at least 80%, 85%, 90%, 95%, 96%, 97%, 98%, 99%, 99.5% or 99.9% identity with SEQ ID NO:1.

As used herein, the term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogues can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid. For example, during chemical synthesis of a synthetic peptide, the native amino acid can be readily replaced by another naturally occurring amino acid or an unnatural amino acid.

As used herein, the term “peptide” corresponds to the chemical agents belonging to the protein family. A peptide is composed of a mixture of several amino acids. Depending on the number of amino acids involved, peptides are categorized as dipeptides, composed of 2 amino acids, tripeptides, made up of 3 amino acids, and so on. Peptides composed of more than 10 amino acids are called polypeptides. Thus, the peptide of the invention can be considered as a polypeptide.

The peptide according to the invention, may be produced by conventional automated peptide synthesis methods or by recombinant expression. General principles for designing and making proteins are well known to those of skill in the art.

Peptides of the invention may be synthesized in solution or on a solid support in accordance with conventional techniques. Various automatic synthesizers are commercially available and can be used in accordance with known protocols as described in Tam et al., 1983; Merrifield, 1986 and Barany and Merrifield, 1979. Peptides of the invention may also be synthesized by solid-phase technology employing an exemplary peptide synthesizer such as a Model 433A from Applied Biosystems Inc. The purity of any given protein; generated through automated peptide synthesis or through recombinant methods may be determined using reverse phase HPLC analysis. Chemical authenticity of each peptide may be established by any method well known to those of skill in the art. As an alternative to automated peptide synthesis, recombinant DNA technology may be employed wherein a nucleotide sequence which encodes a protein of choice is inserted into an expression vector, transformed or transfected into an appropriate host cell and cultivated under conditions suitable for expression as described herein below. Recombinant methods are especially preferred for producing longer polypeptides. A variety of expression vector/host systems may be utilized to contain and express the peptide or protein coding sequence. These include but are not limited to microorganisms such as bacteria transformed with recombinant bacteriophage, plasmid or cosmid DNA expression vectors; yeast transformed with yeast expression vectors (Giga-Hama et al., 1999); insect cell systems infected with virus expression vectors (e.g., baculovirus, see Ghosh et al., 2002); plant cell systems transfected with virus expression vectors (e.g., cauliflower mosaic virus, CaMV; tobacco mosaic virus, TMV) or transformed with bacterial expression vectors (e.g., Ti or pBR322 plasmid; see e.g., Babć et al., 2000); or animal cell systems. Those of skill in the art are aware of various techniques for optimizing mammalian expression of proteins, scc e.g., Kaufman, 2000; Colosimo et al., 2000. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells. Exemplary protocols for the recombinant expression of the peptide substrates or fusion polypeptides in bacteria, yeast and other invertebrates are known to those of skill in the art and a briefly described herein below. U.S. Pat. Nos. 6,569,645; 6,043,344; 6,074,849; and 6,579,520 provide specific examples for the recombinant production of peptides and these patents are expressly incorporated herein by reference for those teachings. Mammalian host systems for the expression of recombinant proteins also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing which cleaves a “prepro” form of the protein may also be important for correct insertion, folding and/or function. Different host cells such as CHO, HeLa, MDCK, 293, WI38, and the like have specific cellular machinery and characteristic mechanisms for such post-translational activities and may be chosen to ensure the correct modification and processing of the introduced, foreign protein.

In the context of the invention, cellulose-bound peptide arrays encompassing the heparin binding domain and the C-terminal human VEGF sequence (UniProtKB #P15692) were synthesized by Proteomic Solutions. Overlapping 15-mer peptides were shifted by 3 aa and two copies of the same array were spotted on the slide for quality control and reproducibility. Arrays were blocked for 2 h in Tris buffered saline, 1% Tween 20, 5% BSA to prevent unspecific binding, and were subsequently probed for 15 h at 4° C. with biotinylated AB42 oligomers (Aβo (42)) using concentrations varying from 0.1 to 10 μg.mL-1 or vehicle used as a control. After washing in TBS 1% Tween 20, peptide arrays were incubated with HRP-conjugated Streptavidin for 2 h at RT. Aβo interaction was detected using SuperSignal West Pico PLUS chemiluminescent substrate and non-specific Aβ binding was determined using the control peptide (CP) spotted on the peptide array. The empty arrowhead indicates the positive control, the biotin, whereas the downward arrowhead points to the negative control, the CP, with the FLAG sequence.

In some embodiments, the invention relates to a nucleic acid encoding an amino acid sequence comprising SEQ ID NO: 1. Nucleic acids of the invention may be produced by any technique known per se in the art, such as, without limitation, any chemical, biological, genetic or enzymatic technique, either alone or in combination(s).

As used herein, the term “protein” refers to any organic compounds made of amino acids arranged in one or more linear chains (also referred as “polypeptide chains”) and folded into a globular form. It includes proteinaccous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecule, etc.,

The term “recombinant protein”, as used herein, includes proteins that are prepared, expressed, created or isolated by recombinant means, such as fusion proteins isolated from a host cell transformed to express the corresponding protein, e.g., from a transfectoma, etc.,

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. A protein fusion of the present disclosure thus includes at least one of R-spondin 1 polypeptide or a fragment or variant thereof as described below, and at least one other moiety, the other moiety being a polypeptide other than a R-spondin 1 polypeptide or functional variant or fragment thereof. In certain embodiments, the other moiety may also be a non protein moiety, such as, for example, a polyethyleneglycol (PEG) moiety or other chemical moiety or conjugates. The second moiety can be a Fc region of an antibody, and such fusion protein is therefore referred as a «Fc fusion protein».

In another embodiment, the invention relates to an expression vector comprising a nucleic acid sequence encoding an amino sequence comprising SEQ ID NO: 1. According to the invention, expression viral vectors suitable for use in the invention may be used.

In a particular embodiment, the peptide according to the invention, wherein the viral vector is adenovirus.

As used herein, the term “adenovirus” refers to medium-sized (90-100 nm), nonenveloped (without an outer lipid bilayer) viruses with an icosahedral nucleocapsid containing a double stranded DNA genome.

In a particular embodiment, the peptide according to the invention, wherein the viral vector is an adeno-associated virus (AAV) vector.

As used herein the term “AAV” has its general meaning in the art and is an abbreviation for adeno-associated virus, and may be used to refer to the virus itself or derivatives thereof. The term covers all serotypes and variants both naturally occurring and engineered forms. According to the invention the term “AAV” refers to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8) and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_001401 (AAV-2), AF043303 (AAV-2), and NC_006152 (AAV-5). As used herein, a “rAAV vector” refers to an AAV vector comprising the polynucleotide of interest (i.e the polynucleotide encoding for the peptide). The rAAV vectors contain 5′ and 3′ adeno-associated virus inverted terminal repeats (ITRs), and the polynucleotide of interest operatively linked to sequences, which regulate its expression in a target cell.

In a particular embodiment, the peptide according to the invention, wherein the AAV vector is selected from vectors derived from AAV serotypes having tropism for and high transduction efficiencies in CNS targeting.

In a particular embodiment, the peptide according to the invention, wherein the AAV vector is an AAV1, AAV2, AAV3, AAV4, AAV 5, AAV 6, AAV7, AAV 8 or AAV9.

In a particular embodiment, the peptide according to the invention, wherein the AAV vector is an AAV9-PhP.B.

In a particular embodiment, the peptide according to the invention, wherein the AAV vector is an AAV9.

In particular, the AAV9 and AAV9-PhP.B variant may be used for their most efficient delivery and transduction across the BBB. The AAV9-PHP.B variant is generated by inserting the sequence encoding the PHP.B peptide (TLAVPFK) in the wild-type AAV9 capsid sequence. AAV vectors may be generated by packaging a recombinant genome or a self-complementary recombinant genome in AAV9 or AAV9-PhP.B capsid. by including the cDNA nucleic acid sequence encoding the amino sequence comprising SEQ ID NO: 1 cloned into an AAV2-based expression cassette containing Enhancer/Promoter combination elements, such as but not limited to the CMV enhance r/β-actin (CB) promoter combination or the CMV early enhancer/chicken β-actin (CAG) promoter.

In a particular embodiment, the peptide according to the invention, wherein the AAV vector is an AAV2.

In a particular embodiment, the peptide according to the invention, wherein the AAV vector is an AAVrh10.

The expression vectors comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system.

It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

In some embodiments, the invention relates to a host cell comprising the expression vector as descried above. Examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art. In another embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

### Therapeutic Method

Inventors show that the peptide as defined above binds to Aβ oligomers with high affinity and inhibits the process of Aβ self-aggregation leading to the blockade of fibrillar aggregation.

Furthermore, the peptide prevents soluble Aβ-derived toxins to target synapses in hippocampal neuron cultures. In addition, it rescues long-term potentiation (LTP) in the APP/PS1 mouse model of Alzheimer's disease. Thus, these findings have broad implications for preventing and treating diseases with Aβ neurotoxicity such as Alzheimer's disease.

Accordingly, in a second aspect, the invention relates to a method for treating a subject suffering from neurodegenerative disorder comprising a step of administering said subject with a therapeutically effective amount of an inhibitor of interaction between amyloid-beta oligomers (Aβo) and vascular endothelial growth factor (VEGF).

As used herein, the term “neurodegenerative disorder” refers to an umbrella term covering a range of conditions caused by age, disease, trauma or combinations thereof, which primarily affect neurons in the human brain and spinal cord. These neurons are the building blocks of the nervous system, and, unlike many other cell types, they normally don't reproduce or replace themselves. In the context of the invention, the neurodegenerative disorder refers to all neurodegenerative diseases showing an accumulation of aggregated amyloid-β (AB).

In a particular embodiment, the neurodegenerative disease is selected from the group consisting of but not limited to: Alzheimer's disease and related disorders, Cerebral amyloid angiopathy (CAA), Down syndrome, Parkinson's disease and related disorders, motor neuron diseases, Frontotemporal dementia (FTD), neuro-inflammatory diseases, Amyotrophic lateral sclerosis (ALS) and Frontotemporal lobar degeneration (FTLD).

In a particular embodiment, the neurodegenerative disorder is selected from the group consisting of but not limited to: Alzheimer disease (AD), Cerebral amyloid angiopathy (CAA), Down syndrome, Parkinson disease and Amyotrophic lateral sclerosis (ALS).

As used herein, the term “subject” denotes a mammal, such as a rodent, a feline, a canine, and a primate. Particularly, the subject according to the invention is a human. More particularly, the subject according to the invention has or is susceptible to have a neurodegenerative disorder.

In a particular embodiment, the subject according to the invention has or is susceptible to have Alzheimer disease (AD).

In a particular embodiment, the subject according to the invention has or is susceptible to have Cerebral amyloid angiopathy (CAA).

In a particular embodiment, the subject according to the invention has or is susceptible to have Down syndrome.

In a particular embodiment, the subject according to the invention has or is susceptible to have Parkinson disease.

In a particular embodiment, the subject according to the invention has or is susceptible to have Huntington disease or Amyotrophic lateral sclerosis (ALS).

As used herein, the term “amyloid-beta oligomers” (Aβo) refers to multimer species of Aβ monomer that result from self-association of monomeric species. Aβ oligomers are predominantly multimers of Aβ1-42, although Aβ oligomers of AB1-40 have been reported. Aβ oligomers may include a dynamic range of dimers, trimers, tetramers and higher-order species following aggregation of synthetic Aβ monomers in vitro or following isolation/extraction of Aβ species from human brain or body fluids.

As used herein, the term “Vascular endothelial growth factor” (VEGF) also known as vascular permeability factor (VPF) refers to a canonical angiogenic factor. VEGF is produced by many cell types including tumor cells, macrophages, platelets, keratinocytes, renal mesangial cells and neural cells such as neurons, glial cells or neural stem and progenitor cells. The activities of VEGF are not limited to the vascular system; VEGF plays a role in normal physiological functions such as bone formation, hematopoiesis, wound healing, brain development and processes occurring in the adult brain such as adult neurogenesis, synaptic plasticity, learning and memory.

As used herein, the term “inhibitor of the interaction between amyloid-beta oligomers (Aβo) and vascular endothelial growth factor (VEGF)” refers to a natural or synthetic compound that has a biological effect to inhibit or significantly reduce the interaction between Aβo and VEGF.

In a particular embodiment, the method according to the invention, wherein the inhibitor mimics a specific domain of the VEGF protein which is targeted by Aβo.

In a particular embodiment, the method according to the invention, wherein the inhibitor targets Aβo with high affinity.

In a particular embodiment, the method according to the invention, wherein the inhibitor prevents the formation of toxic Aβo.

In a particular embodiment, the method according to the invention, wherein the inhibitor prevents the formation of Aβ aggregates and fibrils.

Typically, the inhibitor of the interaction between Aβo and VEGF is a peptide, a polypeptide, a small organic molecule, an aptamer or an antibody.

In a particular embodiment, the method according to the invention, wherein the inhibitor is a peptide as described above.

In a particular embodiment, the method according to the invention, wherein the inhibitor is a peptide comprising or consisting of the amino acid sequence SEQ ID NO: 1:

Accordingly, the invention relates to a peptide comprising the amino acid sequence KRKKSRYKSWSVYVG (SEQ ID NO: 1) for use in the treatment of a neurodegenerative disorder.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is mean the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient (inhibitor of the interaction between Abeta and VEGF) for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

As used herein the terms “administering” or “administration” refer to the act of injecting or otherwise physically delivering a substance as it exists outside the body (e.g., an inhibitor of the interaction between Aβo and VEGF, e.g. a peptide) into the subject, such as by intraparenchymal, intracerebroventricular, intrathecal, intranasal, mucosal, intradermal, intravenous, subcutaneous, intramuscular delivery, oral and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

In a particular embodiment, the invention relates to a method of treating a subject suffering from a neurodegenerative disorder comprising a step of administering said subject with i) an inhibitor of the interaction between amyloid-beta oligomers (Aβo) and the vascular endothelial growth factor (VEGF) and ii) a classical treatment as a combined preparation for simultaneous, separate or sequential use.

In a particular embodiment, the method according to the invention wherein the inhibitor of interaction between amyloid-beta oligomers (Aβo) and the vascular endothelial growth factor (VEGF) is a peptide comprising or consisting of the amino acid sequence SEQ ID NO: 1 as described above.

As used herein, the term “classical treatment” refers to treatments well known in the art and used to treat neurodegenerative disorder. In the context of the invention, the classical treatment refers to acetylcholinesterase inhibitors, N-methyl-D-aspartate (NMDA) receptor antagonist, anti-Aβ antibodies and tyrosine kinase inhibitors.

In a particular embodiment, the method according to the invention wherein the classical treatment is selected from the group consisting of but not limited to: acetylcholinesterase inhibitors such as tacrine, donepezil, rivastigmine, galantamine; N-methyl-D-aspartate (NMDA) receptor antagonist such as memantine.

In a particular embodiment, the classical treatment is an PRX012 (an anti-Amyloid Beta Antibody FDA approved for fast track designation).

In a particular embodiment, the classical treatment is Aducanumab (an anti-Amyloid Beta Antibody FDA approved).

In a particular embodiment, the classical treatment is Masitinib (a tyrosine kinase inhibitors recently in a Phase 2B/3 clinical trial).

In a particular embodiment, the invention relates to i) an inhibitor and ii) a classical treatment used as a combined preparation for simultaneous, separate or sequential use in the treatment of neurodegenerative disorder.

As used herein, the term “simultaneous use” refers to administration of 2 active ingredients by the same route and at the same time or at substantially the same time. The term “separate use” refers to an administration of 2 active ingredients at the same time or at substantially the same time by different routes. The term “sequential use” refers to an administration of 2 active ingredients at different times, the administration route being identical or different.

### Pharmaceutical Composition

In a third aspect, the invention relates to a pharmaceutical composition comprising the inhibitor of the interaction between amyloid-beta oligomers (Aβo) and vascular endothelial growth factor (VEGF).

In a particular embodiment, the invention relates to a pharmaceutical composition, wherein the inhibitor is a peptide comprising or consisting of the amino acid sequence SEQ ID NO: 1.

In a particular embodiment, the invention relates to a pharmaceutical composition according to the invention, wherein the peptide comprising or consisting of the amino acid sequence SEQ ID NO: 1 is inserted or not into a vector.

In a particular embodiment, the invention relates to a pharmaceutical composition comprising i) the inhibitor of the interaction between amyloid-beta oligomers (Aβo) and the vascular endothelial growth factor (VEGF); and ii) a classical treatment as a combined preparation for simultaneous, separate or sequential use in the treatment of neurodegenerative disorder.

The inhibitor of the interaction between Aβo and VEGF according to the invention (or the vector comprising the peptide) may be combined with pharmaceutically acceptable excipients, and optionally sustained-release matrices, such as nanoparticles composed of biodegradable polymers but not limited to polyethylene glycol (PEG), polylactic acid (PLA), polyglycolic acid (PGA), poly lactic-co-glycolic acid (PLGA), poly (methyl methacrylate) (PMMA)), or nanoparticles composed of magnetic compound (such as iron oxide NPs), lipid-based nanoparticle, polymeric or lipid-based micelles, or liposomes, to form pharmaceutical compositions. These sustained-release matrices may be further functionalized either by covalent or non-covalent conjugation using but not limited to Polyethylene glycol, Dextran or Chitosan.

As used herein, the terms “pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The peptide or the drug conjugate (or the vector comprising peptide or the drug conjugate) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject. The drug conjugate (or the vector containing the drug conjugate) may be formulated within a therapeutic mixture to comprise about 0.0001 to 1.0 milligrams, or about 0.001 to 0.1 milligrams, or about 0.1 to 1.0 or even about 100 milligrams per dose. Multiple doses can also be administered. The invention will be further illustrated by the following figures and examples.

### Method of Screening

In another aspect, the present invention relates to a method of screening an inhibitor of the interaction between Amyloid-beta oligomers (Aβo) and VEGF suitable for targeting Aβo and for the treatment of a neurodegenerative disorder comprising i) providing a test compound and ii) determining the ability of said test compound to inhibit and/or reduce the interaction between Aβo and VEGF.

In a particular embodiment, the method according to the invention, wherein such inhibitor mimics a specific domain of the VEGF protein which is targeted by Aβo.

In a particular embodiment, the method according to the invention, wherein the inhibitor prevents Aβ aggregation and the formation of toxic Aβo.

In a particular embodiment, the method according to the invention, wherein the inhibitor interferes with synaptic targeting by toxic Aβo.

More particularly, the method according to the invention, wherein the inhibitor rescues synaptic plasticity impaired in the APP/PSI mouse model of Alzheimer's disease.

Any biological assay well known in the art could be suitable for determining the ability of the test compound to inhibit and/or reduce the interaction between Amyloid-beta oligomers (Aβo) and VEGF.

In some embodiments, the assay comprises determining the ability of the test compound to target Aβo and interfere with Aβo toxicity. In some embodiments, a population of Aβ producing cells is then contacted and activated so as to determine the ability of the test compound to interact with Aβo and to inhibit Aβ aggregation.

In particular, the effect triggered by the test compound is determined relative to that of a population of cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of targeting Aβo and of inhibiting and/or reducing their toxicity, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. Selective binding of Aβo to the VEGF Heparin Binding Domain and identification of a potent binding site. Representative peptide array showing Aβ interacting sites in the heparin binding domain of the VEGF protein sequence with various concentrations of biotinylated Aβo or vehicle (control). Note the strong labeling of the fifth spot corresponding to the blocking peptide sequence indicated by the upward arrowhead, even at a low Aβo concentration. The positive control corresponds to the Biotin sequence indicated by the empty arrowhead. In contrast no labeling was observed in the control condition when incubation was performed with the vehicle alone (except for the positive control spot). Similarly, no labelling was obtained for the control peptide corresponding to the FLAG sequence (NDYKDDDDKGAAA) and indicated by the downward arrowhead, at any of the Aβo concentrations.

FIG. 2. Blocking peptide (BP) inhibits Aβo-VEGF interaction and specifically binds to Aβo with high affinity. A) Dose-dependent competitive ELISA showing the inhibition of Aβ binding to VEGF by the BP (triangle) and not by the CP (filled circle). B,C) Dose-dependent binding of biotinylated forms of Aβ42 oligomers (Aβo) (B) or AB40 monomers (Aβm) (C) to the blocking (BP) or control peptides (CP) analyzed by ELISA. Titration binding curves show that the binding affinity of the BP for Aβo (B) is much greater than for Aβm (C). No binding of the CP is detected to either Aβo (B) or Aβm (C). n=5 from 5 independent experiments.

FIG. 3. BP blocks Aβ aggregation and fibrillation. A,B) Kinetics of Aβ aggregation and fibril formation analyzed by ThT fluorescence using 15 μM of monomeric AB42 with increasing concentrations of the CP or BP (from 0 to 15 μM shown with dotted lines). Each curve represents the normalized time course with data plotted every 5 minutes up to 24 h, showing the mean of 4 independent experiments. Note the concentration dependent inhibitory effect of the BP on Aβ aggregation and fibrillization (B). C,D) Quantitative analyses of Aβ aggregation performed at 24 h demonstrating that at equimolar concentration the BP prevents Aβ aggregation (D) in contrast to the CP (C).*** p<0.001, Kruskal-Wallis followed by Dunn post-hoc test, n=4 from 4 independent experiments.

FIG. 4. BP inhibits Aβ fibril formation and promotes amorphous aggregates production. A) TEM photomicrographs illustrating extended fibrils formed after 24 hours in the Aβ condition alone (30 μM) that display a characteristic twisted morphology. B,C) Similar fibrillar structures with a twisted morphology were obtained when Aβ is incubated in the presence of the CP at an equimolar concentration (30 μM). D) Aβ aggregates produced in the presence of the BP are amorphous structures with occasional fibrillar content, showing a range of different sizes. E,F) Negatively stained samples of the CP (E) and BP (F) do not show any detectable 3D structures by TEM. Scale bars, A, B, 200 nm; C, D, E, F 100 nm.

FIG. 5. Reduction in HMW soluble Aβo species when Aβ aggregated with the BP. Aβ was aggregated in the presence of the CP or BP for 2 or 24 hours, and Aβ species were analyzed by Western Blotting using the 6E10 anti-Aβ antibody. The distribution of soluble Aβ species including Aβ monomers (1-mer), LMW and HMW Aβo was quantitatively assessed. A) Quantitative analysis showing a significant decrease in HMW Aβo together with an increase in LMW Aβo when Aβ aggregated with the BP for 2 hours. B) A similar decrease in HMW Aβo is observed after 24 h of aggregation with the BP compared to the other conditions. In contrast, both the Aβ monomers and LMW Aβo increase in the presence of the BP compared to the other conditions.* p<0.05, ** p<0.01, Kruskal-Wallis followed by Dunn post-hoc test, n=5 from 5 independent experiments.

FIG. 6. Inhibition of Aβ synaptic targeting in the presence of the BP. A,B) Confocal images of DIV 21 primary hippocampal neurons showing immunostained synapses with colocalization of PSD95 and Bassoon reflecting full synapses (white). Cell surface labeling of biotinylated Aβ (white) along neuronal processes is illustrated in the same Aβ-treated hippocampal neurons. A) Note the colocalization of biotinylated Aβ (right panels) with PSD95 and Bassoon positive synapses (left panels) when hippocampal neurons were treated with HMW Aβo enriched preparations formed in the presence of the CP. B) In contrast, biotinylated Aβ binding to hippocampal cell surface and synapses is greatly reduced when treatment are performed with HMW Aβo enriched preparations formed in the presence of the BP. Scale bar, 10 μm. n=8-9 from 3 independent experiments.

FIG. 7. Acute BP treatment rescues LTP in APP/PS1 mice at 8 months. Time course of LTP experiments in hippocampal slices from 8-month-old WT (A, C, E) and APP/PS1 mice (B, D, F), and after administration of the CP (C, D) or BP (E, F). A) A robust LTP was induced by theta burst stimulation (TBS) in slices of WT mice (empty circles, n=6 micc). B) In contrast, LTP was considerably reduced in APP/PSI slices (filled circles, n=7 micc). C, E) Addition of the CP (C, n=5 micc) or BP (E, n=6 micc) does not alter LTP responses in WT slices. D, F) When the CP was administrated to APP/PSI slices (D, n=5 mice), TBS failed to increase LTP responses up to 1 h after TBS. In contrast, the BP induced a progressive and sustained increase in responses characteristic of LTP 1 h after TBS (F, n=5 mice), G) Summary bar graph showing that LTP is decreased in APP/PSI slices incubated with the CP compared to WT slices incubated with the CP (LTP amplitude computed as fEPSP slope 50 to 60 min after TBS is decreased from 191.4±18.3% in WT slices+CP to 119.8±9.1% in APP/PSI slices+CP, n=7-6, * (Kruskal Wallis test (p=0.01) followed by Dunn post hoc test, p<0.05). In contrast, treatment with the BP rescued LTP in the APP/PS1 slices compared to the CP condition (LTP is increased from 119.8±9.1% in APP/PSI slices+CP to 226.7±40.5% in APP/PSI slices+BP, n=7-7, * p<0.05).

### EXAMPLE

### Material & Methods

Animals specimens

Animals specimens Embryonic day 17-18 (E17-18) C57BI/6JRj wild-type male and female mice were used for primary hippocampal cell cultures. Electrophysiological field potential recording experiments were performed on 8-month-old wild type and transgenic heterozygous male APP/PS1-21 mice generated on a C57Bl/6 background and expressing a transgene combining human APP with the Swedish mutation (APPKM670/671NL) and mutated L166P human presenilin 1 (PS1) under the Thyl promoter (Radde et al, 2006). Wild type and APP/PSI mice were generated as described previously (Radde et al, 2006) and genotyping was carried out to reveal the presence or absence of APP and PSI transgenes. The study was conducted in accordance with the European Community Council directive 2010/63/EU on the protection of animals used for experimental and scientific purposes. Animal care and treatment procedures were realized according to the guidelines approved by the French Ethical Committee of the Lyon 1 University (DR2013-47).

### Mouse Primary Hippocampal Neuronal Culture

Primary hippocampal neuron cultures were prepared from E17-18 C57Bl/6JRj mice. Briefly, hippocampi were removed, cut into pieces in Hank's buffered salt solution (HBSS), digested in HBSS supplemented with trypsin (0.25% v/v) and rinsed in HBSS and BSA 0.2%. Next, hippocampi were triturated in Neurobasal medium without phenol red supplemented with L-glutamine (2 mM), 2% B27, 1% penicillin-streptomycin (10000 U/mL). Hippocampal neurons were then plated onto poly-L-lysine coated coverslips (0.5 mg/ml) at a low density (15×103 cells/cm2) for synaptic targeting experiments. Neurons were subsequently cultured for 21 DIV in supplemented Neurobasal medium at 37° C. under 5% CO2, one-half of the media being changed once a week.

### Hippocampal Neuron Treatment

For synaptic targeting experiments, hippocampal neuron cultures were used after 21 days in vitro (DIV) and treated for 30 min with 500 nM biotinylated Aβ preparations enriched in HMW Aβo. HMW Aβo enriched preparations are derived from Aβm which have been incubated at 15 μM in the presence or not of equimolar concentration of the CP or BP for 2 h at 37° C.

### Hippocampal Neuron Immunostaining

Treated hippocampal neuron cultures were fixed with 4% paraformaldehyde in 0.1 M phosphate buffer at RT prior to immunostaining. After a blocking step in non-permeabilizing condition (PBS, 1% BSA) surface-bound biotinylated Aβ was detected with fluorescently tagged streptavidin (Alexa Fluor 647, Invitrogen) incubated for 3 h at RT. Three washes were performed next followed by a permeabilizing step in PBS, 0.3% Triton-X-100 and 1% BSA for 1 h, prior to an overnight incubation at 4° C. with antibodies directed against PSD95 and Bassoon in the permeabilizing blocking solution. Subsequently, appropriate Alexa conjugated secondary antibodies were incubated for 2 hours at RT and cultures were rinsed, counterstained with DAPI and mounted in Fluoromount reagent.

Confocal image acquisition and analysis

Immunostainings images were obtained using a confocal Zeiss LSM 880 AiryScan microscope equipped with two diode lasers (405, 561 nm), a laser gaz argon (488 nm) and a laser gaz He/Ne (633 m), a 63x objective and an additional zoom factor (3x). For image acquisition identical acquisition parameters were used between treated conditions by an investigator blinded to cell culture treatments and 3D confocal z-stack images were deconvolved using Huygens software. Subsequent analyses were performed by a blinded investigator with Icy software. To assess biotinylated Aβ expression at full synapses, regions of interest (ROIs) used to identify synapses were defined as the colocalization area between PSD95 and Bassoon positive clusters on the dendrite of hippocampal neurons. Biotinylated Aβ clusters were quantified in each ROI in collapsed Z-stacks.

Aβ preparations

Synthetic AB40, Aβ42, and their biotinylated forms were obtained as lyophilized samples from Bachem (AB42, b-AB40, AB42 and b-Aβ42). Briefly, peptides were solubilized in 1,1,1,3,3,3-hexafluoro-2-propanol to prevent oligomerization, then evaporated overnight under a chemical fume hood, and stored as a dried peptide film at −80° C. until use, as previously described (Stine et al. 2003). Aβ monomers (Aβm) were prepared extemporancously by first dissolving the peptide film in 2 mM dimethyl sulfoxide with an additional dilution step to 100 μM in ice-cold PBS. Diluted peptides were subsequently sonicated for 20 min at 4° C. and centrifuged at 10000 g for 3 minutes, the supernatant was collected and concentration of Aβm measured using a micro BCA protein assay (ThemoFisher) prior to being aliquoted and stored at −20° C. until use. Typically, this centrifugation step resulted in the loss of around 50 to 60% of the initial Aβ amount. Next, we prepared Aβ42 peptides in different oligomerization states, one enriched in high molecular weight Aβ oligomers (HMW Aβo) and the other one in low molecular weight Aβ oligomers (LMW Aβo). To prepare Aβ preparations enriched in HMW Aβo, monomeric AB42 peptides (Aβm) were incubated at a concentration of 15 μM during 2 hours at 37° C. For synaptic targeting experiments, HMW Aβo enriched preparations were administrated to hippocampal cultures to a final concentration of 500 nM in appropriate culture medium. For electron microscopy experiments, we used HMW Aβo enriched preparations at 30 μM. In a subset of experiments (ELISA assays), we also used Aβo enriched in LMW Aβo. In this case, Aβm was incubated for 24 h at 4° C., centrifuged at 10000 g for 10 minutes and the supernatant was collected as LMW Aβo. For ELISA assays, LMW Aβo enriched preparations were used at concentrations varying from to from 0.1 μg.mL-1 to 10 μg.mL-1.

In all the experiments Aβo concentrations are expressed as monomer equivalent concentrations, as previously reported (Lauren et al. 2009).

### Peptide Arrays

Cellulose-bound peptide arrays encompassing the heparin binding domain and the C-terminal human VEGF sequence (UniProtKB #P15692) were synthesized by Proteomic Solutions. Overlapping 15-mer peptides were shifted by 3 aa and two copies of the same array were spotted on the slide for quality control and reproducibility. Arrays were blocked for 2 h in Tris buffered saline, 1% Tween 20, 5% BSA to prevent unspecific binding, and were subsequently probed for 15 h at 4° C. with biotinylated Aβ42 oligomers (Aβo (42)) using concentrations varying from 0.1 to 10 μg.mL-1 or vehicle used as a control. After washing in TBS 1% Tween 20, peptide arrays were incubated with HRP-conjugated Streptavidin for 2 h at RT. Aβo interaction was detected using SuperSignal West Pico PLUS chemiluminescent substrate and non-specific Aβ binding was determined using the control peptide (CP) spotted on the peptide array. The empty arrowhead indicates the positive control, the biotin, whereas the black arrowhead points to the negative control, the CP, with the FLAG sequence.

### ELISA Assays

The binding of the CP or BP to synthetic biotinylated Aβ42 oligomers (Aβo) or AB40 monomers (Aβm) was determined by indirect ELISA in which the CP or BP were used as the capture antigens and the biotin tag of the biotinylated Aβ peptides for the detection. 96-well clear polystyrene microplate (BioTechne DY990) were coated overnight at 4° C. with 5 μM of the CP or BP in PBS, pH 7.4. After immobilization, the plate was washed three times with PBS 0.05% Tween 20 and various concentrations of biotinylated Aβ peptides were added in triplicate from 0.1 μg.mL-1 to 10 μg.mL-1 (21 nM to 2.1 μM for Aβo (42), 22 nM to 2.2 μM for Aβm (40) and incubated for 2 h at RT. After additional washes with PBS 0.05% Tween 20, HRP-conjugated streptavidin (1/40) was added for 20 min at RT, followed by a washing step and an incubation in substrate solution containing 3,3′,5,5′-Tetramethylbenzidine (TMB) and H₂O₂. The reaction was stopped by H2SO4. Absorbance was successively measured at 450 and 540 nm with a TECAN microplate reader and optical density values at 540 nm were subtracted from the ones at 450 nm to correct for optical imperfections of the plate. Unspecific Aβ binding to the microplate was determined using non-coated wells.

Competitive ELISA experiments were performed to analyze the ability of the BP to impede AB42 oligomers binding to VEGF. For these assays, recombinant human VEGF165 (500 ng/ml-1) was immobilized in the 96-well microplate as the capture antigen and incubated overnight at 4° C. The following day, biotinylated AB42 oligomers were precubated at 2.1 μM for 1 h at RT with various concentrations of the CP or BP ranging from 10 nM to 100 μM. After 3 rinces with PBS 0.05% Tween 20, the mix was added in triplicate and incubated for 2 h at RT. Next, a washing step was performed followed by an incubation with the HRP-conjugated streptavidin for 20 min, additional rinces and finally the substrate solution containing TMB and H₂O₂. Absorbance was measured using a TECAN microplate reader.

### Aggregation Kinetics Using Thioflavin T Assays

Thioflavin-T (ThT) assays were performed in black polystyrene 384-well plates with transparent bottom using a Tecan microplate reader with a set of excitation/emission wavelengths of 450/490 nm. Monomeric Aβ42 was prepared as described above and blocking or control peptides (BP ou CP) were used at indicted concentrations, ranging from 0.5 to 15 μM. The final concentration of ThT and monomeric AB42 was used at 15 μM in PBS buffer (pH 7.4), with a total reaction volume per well of 110 μL. The plate was sealed with a transparent plastic film and incubated at 37° C. prior to fluorescence measurement. ThT fluorescence intensity was monitored every 5 min over 24 h at 37° C. without agitation. Three replicates per condition were measured.

### Electron Microscopy

Prior to imaging, 10 μL of an Aβ preparation (30 μM) aggregated for 24 h at 37° C. in the presence or absence of equimolar concentration of the CP or BP were deposited onto Formvar-C coated mesh nickel grids. After a 10 min incubation step at RT, grids were rinsed with sterile dH20. Samples were subsequently stained using 5% uranyl acetate for 1 minute in the dark to increase contrast and were allowed to air dry for 2 minutes. Finally, they were visualized with a JEM 1400 Transmission Electron Microscope (Jcol Tokyo, Japan), operating at 120 kV, initially imaged at low magnification (20,000X), and thereafter at 120,000X using a Gatan Orius 1000 digital micrograph software.

### Electrophysiology

Local field potential recordings (LFP) were performed on acute coronal hippocampal slices from 8-month-old wild type and transgenic heterozygous male APP/PS1-21 mice to measure baseline synaptic response and long-term potentiation (LTP) of Schaffer collaterals to CAI pyramidal cell synapses. Hippocampal slices (400 μm thick) were cut using a vibratome (Leica VT1200S) and incubated at room temperature in artificial cerebrospinal fluid (ACSF) containing (in mM): 124 NaCl, 10 glucose, 1.25 NaH2PO4, 2.5 KCl, 26 NaHCO₃, 1.3 MgCl2, and 2.5 CaCl2, bubbled with 95% 02 and 5% CO2, pH 7.4) for 1 h prior to recording. Field excitatory postsynaptic potentials (fEPSPs) were recorded extracellularly in stratum radiatum of CAI region of the dorsal hippocampus in presence of gammaaminobutyric acid (GABA)-A receptor antagonist picrotoxin (100 μM). Electrical stimulation was realized with a bipolar tungsten electrode placed in CAI stratum radiatum and fEPSPs were measured using borosilicate glass microelectrodes (˜1-3 M (2) filled with ACSF. The tip of the recording electrode was located close to the slice surface. LFP were amplified and low-pass filtered at 3 kHz using a differential amplifier (X1000, WPI) and data acquisition and analyses were carried out using a National Instrument interface coupled with Elphy software (G. Sadoc, CNRS, France). LFP were sampled at 10 kHz and fEPSPs initial slope was computed to quantify synaptic responses. After baseline recordings of synaptic activity evoked at 0.2 Hz for at least 10 minutes, slices were incubated in ACSF or treated with the CP or BP at 500 nM for 40 min before inducing LTP with the theta burst stimulation (TBS). The effects of the peptides on synaptic responses were tested in a series of pilot experiments on mice of both genotypes. The 500 nM concentration of BP did not induce a change in these responses during the 40 min prior to LTP induction. TBS consisted of 10 trains separated by 30 s, each train composed of 6 bursts at 5 Hz and each burst providing 4 pulses at 100 Hz. Before LTP induction, precautions were taken to ensure that approximately the same amplitude of fEPSPs was obtained in baseline for the different experiments to achieve the same level of cooperativity in each group. After LTP induction, fEPSPs were recorded for at least 60 min.

### Immunoblotting

Aβ preparations (1 μg) were subjected to immunoblotting analysis and separated on 4-12% gradient SDS-PAGE gels, prior to being transferred on nitrocellulose membranes. After a 45 minutes blocking step in Tris-buffered saline, 0.1% Tween, pH 7.6, 5% milk, membranes were immunoblotted overnight at 4° C. with the 6E10 anti-Ab antibody diluted in Tris-buffered saline, 0.1% Tween, pH 7.6, with 2% milk. Horseradish peroxidase (HRP)-conjugated secondary anti-mouse antibody was next applied for 1 h30 at room temperature. Proteins were visualized with an ECL detection system and band intensities quantified using a densitometric analysis with Image J software.

### Statistical Analysis

Data were expressed as median with interquartile range in ThT, Western blotting and synaptic targeting experiments and as mean±SEM for electrophysiological recordings. Normality and variance homogeneity were assessed with descriptive statistics and appropriate tests using R and OriginLab softwares. Sample size (n) was determined based on previous studies from the literature and pilot experiments; n refers to the number of Elisa, ThT or Western Blotting experiments, and to mice per condition or per genotype for electrophysiological experiments. For the percentage of Aβ aggregation, Aβ expression level and the percentage of synapses colocalized with AB, data were compared for statistical significance between groups using a Kruskal Wallis and a Dunn's post-hoc test (R software) after checking for normality and homoscedasticity using descriptive analysis and appropriate tests. For electrophysiological data, statistical analysis between treatments and/or genotypes were carried out using a Kruskal Wallis followed by a Dunn's post-hoc test to compare differences in fEPSP slopes.

### Results:

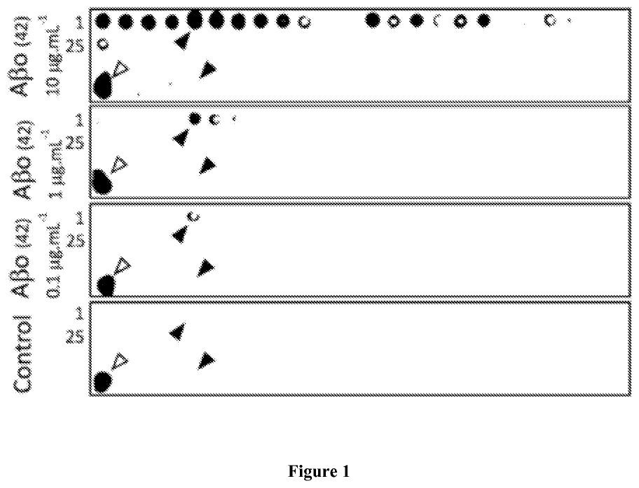

The blocking peptide with the following amino acid sequence, KRKKSRYKSWSVYVG (SED ID NO: 1), was identified using peptide arrays encompassing the heparin binding domain and the C-terminal protein sequence of the human VEGF (FIG. 1). Peptide arrays were incubated in solution of biotinylated oligomeric Aβ42 (Aβo) with concentrations varying from 0.1 μg.mL-1 to 10 μg.mL-1. We observed some heterogencity in the intensities of labelled spots at high Aβo concentration (1 and 10 μg.mL-1) in several potential binding spots of the peptide array. However, at the lowest concentration (0.1 μg/ml-1) only one spot displays a clear staining. The peptide spotted at this specific location (upward arrowhead), named from now on the blocking peptide (BP), has been selected for its ability to bind Aβo and for its potential ability to block Aβo pathogenic effect.

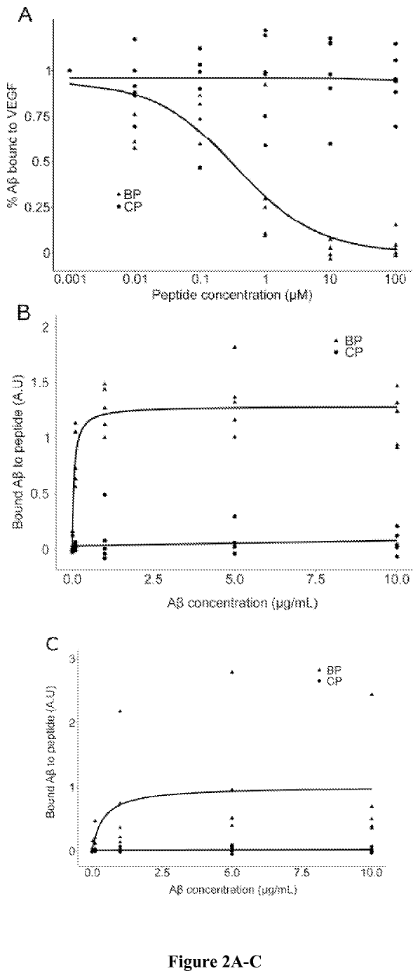

To determine if the BP inhibits the interaction between Aβo and VEGF, we measured biotinylated Aβo binding ability to immobilized VEGF protein in a competitive ELISA assay exposed to increasing BP concentrations. We observed a clear dose-dependent reduction in Aβo-VEGF interaction and assessed the inhibitory constant of the BP to be of 340 nM (FIG. 2A). In contrast, the CP failed to exhibit any effect and did not inhibit the binding of Aβo to VEGF, validating its use as a proper control.

Next, to confirm the BP capacity to efficiently binds Aβo, we used an ELISA-based assay for analyzing its relative binding to oligomeric (Aβo) versus monomeric (Aβm) species using Aβo (42) or Aβm (40) solutions, respectively. ELISA data with titration binding curves revealed that the blocking peptide directly interacts with Aβo with a high binding affinity (KD of 12 nM) but binds monomers much less efficiently (KD of 19 μM) (FIG. 2B,C). In contrast, the control peptide (CP) fails to bind both Aβo and Aβm (FIG. 2B,C). These findings highlight the direct physical interaction between Aβo and the blocking peptide.

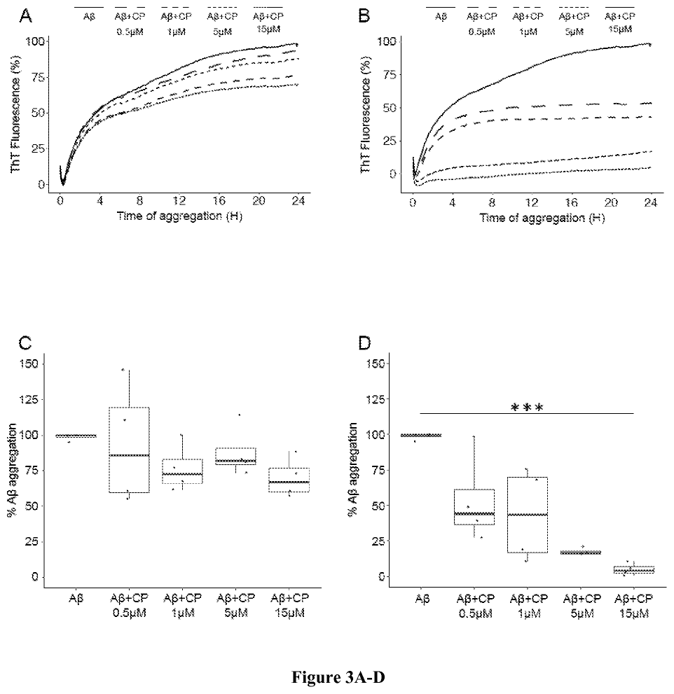

Next, to determine whether the BP could interfere with Aβ aggregation and self-assembly process, which leads to various toxic effects, we performed the gold-standard Thioflavin-T (ThT)-based assay to selectively label Aβ aggregated species and fibrils rich in B sheets. We monitored Aβ aggregation kinetics either in the presence or absence of the CP or BP, based on the correlation between Thioflavin-T fluorescence intensity and amyloid fibril concentration. These experiments allowed us to evaluate the impact of the CP or BP on the aggregation kinetics of AB42, the peptide most prone to self aggregation. We monitored the aggregation of AB42 as a function of time, in presence of various concentrations of the CP or BP ranging from 0.5 to 15 μM (FIG. 3A, B). Next, we normalized the ThT fluorescence levels obtained in each condition to the maximum obtained in the Aβ condition alone. The steepness of the exponential phase corresponding to AB42 nucleation decreases with increasing concentrations of the BP, showing a dose dependent effect (FIG. 3B), in contrast to the experiments performed with the CP (FIG. 3A). Control experiments validated that the highest concentration of the CP or BP alone does not induce any increase in ThT fluorescence and thus no ThT positive aggregates.

To quantitatively compare these data, we considered the normalized ThT fluorescence level obtained after 24 hours in each condition as an index of Aβ aggregation. Incubation of 15 μM of monomeric Aβ in the presence of equimolar concentration of the BP produced a 95% inhibition of fibril formation (FIG. 3D), whereas the CP has no significant effect at the same concentration (FIG. 3C). Here, we demonstrate that the BP but not the CP significantly inhibits Aβ aggregation and fibrillization when used at a concentration cquimolar to AB.

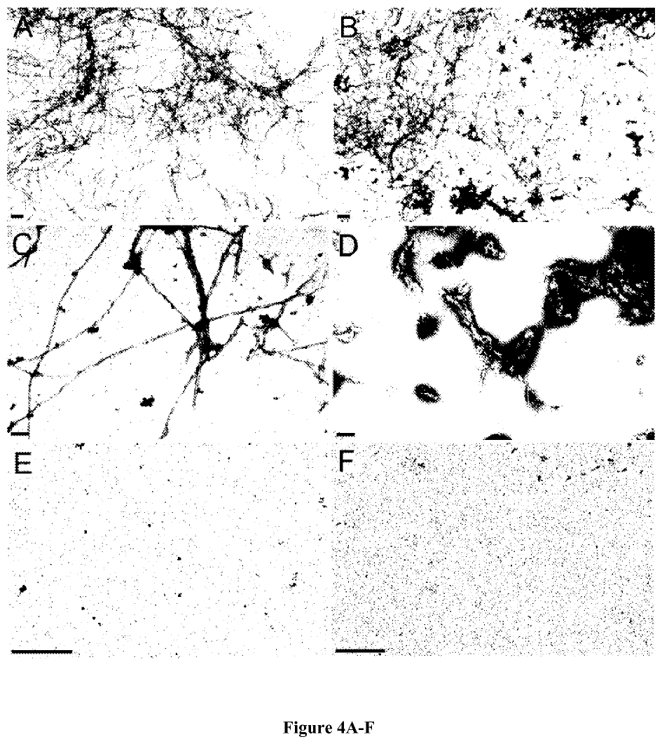

To confirm that the BP can inhibit fibril formation and to gain insight into the size and morphology of the Aβ aggregates formed, we performed a structural analysis using Transmission Electron Microscopy (TEM) after 24 h incubation of Aβ alone or with equimolar concentration of the CP or BP. Typically, Aβ incubated alone forms extensive negatively stained fibrils (FIG. 4A) with a characteristic twisted morphology, also observed when the CP was incubated with Aβ (FIG. 4B, C). In contrast, when Aβ is incubated with the BP, aggregates that are formed are mainly non-fibrillar and amorphous in shape (FIG. 4D), with varying sizes. Control experiments validated that the CP (FIG. 4E) or BP alone (FIG. 4F) does not induce any 3-dimensional self-assembly structures detectable using TEM. Importantly, amorphous Aβ aggregates formed in the presence of the BP are considered as less toxic Aβ species deviating from the aggregation/fibrillation pathway.

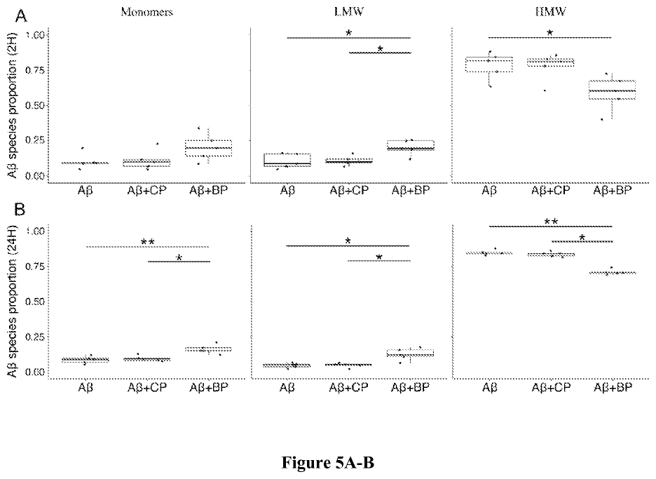

As we previously showed that the BP binds Aβ with high affinity and alters its self-aggregation process leading to the formation of insoluble amorphous aggregates, we further examined its impact on the distribution of soluble Aβ species using a biochemical approach. We analyzed the pool of soluble species at 2 and 24 hours, and compared the fraction of Aβ monomers, low molecular weight (LMW) and high molecular weight (HMW) Aβo amongst the different conditions including Aβ alone, Aβ with the CP or BP. Indeed, it is well known that insoluble fibrils do not enter Western Blotting gel and remain at the top. At 2 hours, a significant decrease in the amount of HMW Aβo was observed in the Aβ and BP condition compared to the Aβ alone condition and a tendency towards a decrease when compared to the Aβ and CP condition (FIG. 5A). In contrast, the fraction of LMW Aβo was significantly increased in the Aβ and BP condition compared to the two other conditions (FIG. 5A). However, this increase in LMW Aβo in the presence of the BP should be considered with caution because it has been previously shown that the presence of SDS in the sample buffer artificially increase the formation of LMW Aβo from the monomeric Aβ pool. At 24 hours, all the conditions but the Aβ and BP condition contain insoluble aggregates at the top of the gel together with Aβ monomers, LMW and HMW Aβo. We observed that only the Aβ and BP condition displays a significant decrease in the fraction of HMW Aβo and an opposite increase in LMW Aβo and Aβ monomers compared to the two other conditions (FIG. 5B). Altogether, we found a clear difference in the relative abundance of Aβo in the presence of the BP with a significant decrease in HMW Aβo.

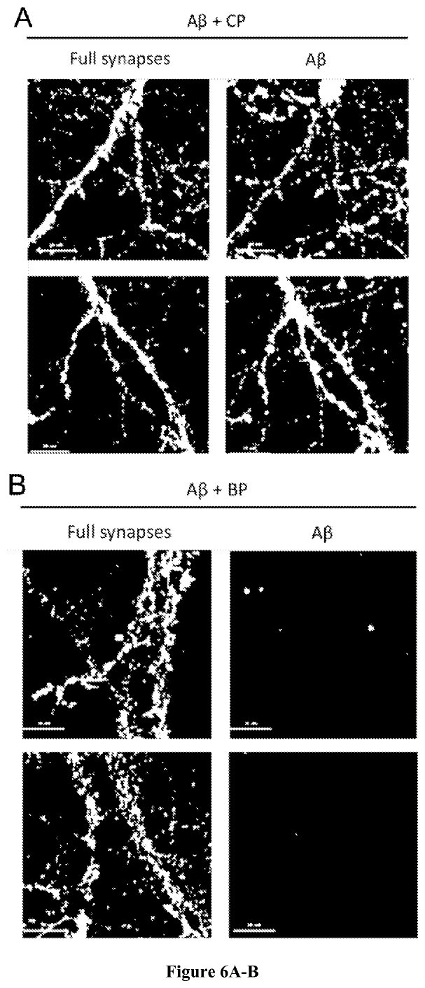

Based on previous reports which demonstrated that it is mainly the HMW Aβo that bind excitatory synapses, we investigated the impact of the CP and BP on Aβo-induced synaptic targeting using immunofluorescence co-localization analyses. We used the 2 h condition of Aβ aggregation that produces a substantial amount of HMW Aβo to treat mature 21 DIV hippocampal neurons for 30 minutes. The binding of biotinylated Aβ to hippocampal cell surface and synapses was examined using streptavidin combined with the use of antibodies recognizing the presynaptic and postsynaptic markers Bassoon and PSD95, respectively. When Aβ was aggregated alone or with the CP, hippocampal neurons exhibit a typical biotinylated Aβ labeling in clustered sites corresponding to synapses positive for Bassoon and PSD95 (FIG. 6A). These data confirm the targeting of hippocampal excitatory synapses by Aβo. Conversely, Bassoon and PSD95 immunoreactive synapses lack biotinylated Aβ positive clusters in most hippocampal neurons in the Aβ and BP condition (FIG. 6B). Thus, cell surface Aβ labeling was impeded when Aβ aggregation is performed in the presence of BP, leading to a clear reduction in synapse targeting by AB. Quantification shows that almost 40% of synapses colocalize with Aβ when Aβ is aggregated alone or with the CP, whereas only a small proportion are targeted by Aβ when Aβ is aggregated with the BP. Thus, the fraction of synapses which bind Aβ in the latter condition is greatly reduced, reaching values equivalent to those in control conditions without Aβ treatment. Overall, these findings indicate that Aβ fail to bind synapses when the BP is present during the Aβ aggregation process. The fact that the BP impedes the ability of Aβo to efficiently target synapses is consistent with our biochemical data showing that the BP markedly affects the formation of HMW Aβo. Taken together, these findings suggest that a threshold in the amount of HMW Aβo might be necessary to efficiently bind synapses and trigger subsequent toxicity.

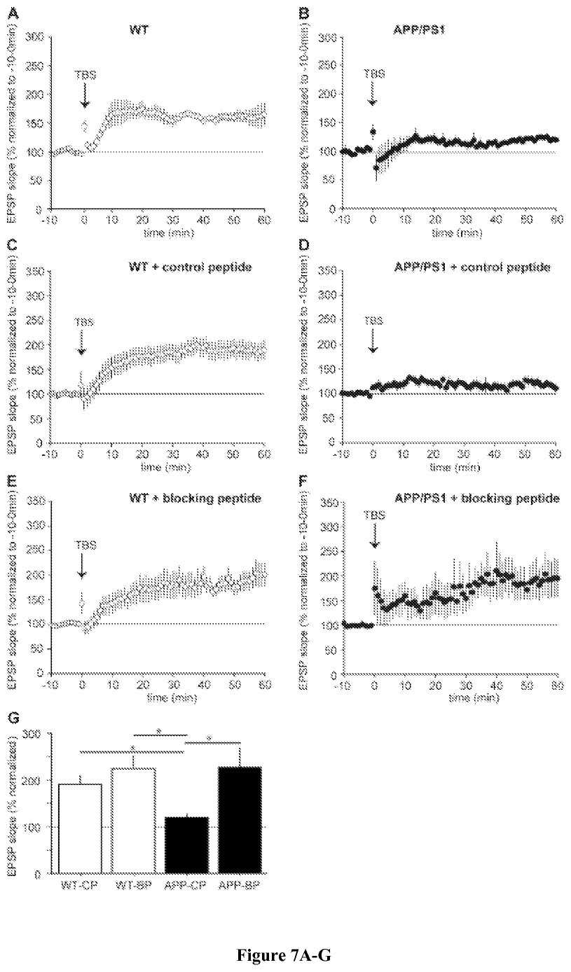

Increasing evidence now demonstrate that Aβo initially target synapses and further inhibit a form of synaptic plasticity considered as the major cellular basis of learning and memory: the long-term potentiation or LTP. Thus, to study functional consequences of BP treatment on synapse function, we performed electrophysiological experiments on acute hippocampal slices from 8-month-old WT and APP/PSI mice, a well-known mouse model of AD, when significant amounts of Aβ plaques are present in the cerebral cortex and hippocampus. Hippocampal slices from both genotypes were treated or not with the CP or BP prior to cliciting a theta-burst stimulation (TBS) of Schaffer collaterals to induce LTP at CA1 pyramidal cell synapses. A large LTP was induced by TBS in WT slices, with a sustained increase in the field excitatory postsynaptic potentials (fEPSP assessed by computing the slope), which is maintained at least for 1 h post-TBS (FIG. 7A). In contrast, LTP was greatly reduced in APP/PSI slices as soon as 5 minutes after TBS and the LTP remained defective at 1 h post-TBS (FIG. 7B). Because we have confirmed that LTP is compromised in APP/PS1 slices, we next examined the potential influence of acute BP treatment on changes in synaptic plasticity. In WT slices, treatment with the CP (FIG. 7C) or BP (FIG. 7E) 40 minutes prior to TBS did not alter LTP compared to the condition without supplementation (FIG. 7A). In contrast, acute treatment of APP/PSI slices with the BP rescued LTP after TBS, which remained sustained at 1 h (FIG. 7F). This restored LTP strongly differs from the one obtained with the CP, which failed to show any substantial increase (FIG. 7E). Thus, even at an age when LTP deficits are pronounced in APP/PSI mice, acute BP application can restore LTP, highlighting the positive effect of the BP treatment on Aβ-mediated synaptic toxicity. These findings indicate that the blocking peptide is a promising candidate for alleviating synaptic defects in AD preclinical models. The next step is now to assess the benefits of this blocking strategy in the APP/PSI mouse model of AD to limit Aβ toxic effect in learning and memory.

### REFERENCES

Babć, L. M., Linnevers, C. J., and Schmidt, B. F. (2000). Production of active mammalian and viral proteases in bacterial expression systems. Biotechnol Genet Eng Rev.17, 213-52.
Barany, G., Merrifield, R. B. (1979). A chromatographic method for the quantitative analysis of the deprotection of dithiasuccinoyl (Dts) amino acids. Anal Biochem. 95, 160-70.
Cao, L., Jiao, X., Zuzga, D. S., Liu, Y., Fong, D. M., Young, D., and During, M. J. (2004). VEGF links hippocampal activity with neurogenesis, learning and memory. Nat. Genet. 36, 827-35.
Chen Y, Wang J, Cui C, Su Y, Jing D, Wu L, Liang P, Liang Z. Evaluating the association between brain atrophy, hypometabolism, and cognitive decline in Alzheimer's disease: a PET/MRI study. Aging (Albany NY). 2021; 13:7228-7246.
Colosimo A, Goncz K K, Holmes A R, Kunzelmann K, Novelli G, Malone R W, Bennett M J, Gruenert D C. (2000). Transfer and expression of foreign genes in mammalian cells. Biotechniques. 29, 314-8, 320-2, 324 passim.
De Rossi, P., Harde, E., Dupuis, J. P., Martin, L., Chounlamountri, N., Bardin, M., Watrin, C., Benetollo, C., Pernet-Gallay, K., Luhmann, H. J., et al. (2016). A critical role for VEGF and VEGFR2 in NMDA receptor synaptic function and fear-related behavior. Mol. Psychiatry 21, 1768-1780.
Dubois, B., Feldman, H. H., Jacova, C., Hampel, H., Molinuevo, J. L., Blennow, K., DeKosky, S. T., Gauthier, S., Selkoe, D., Bateman, R., et al. (2014). Advancing research diagnostic criteria for Alzheimer's disease: the IWG-2 criteria. Lancet Neurol. 13, 614-29.
Giga-Hama, Y., and Kumagai H. (1999). Expression system for foreign genes using the fission yeast Schizosaccharomyces pombe. Biotechnol Appl Biochem. 30, 235-44.
Ghosh, S., Parvez, M. K., Banerjee, K., Sarin, S. K., and Hasnain, S. E. (2002). Baculovirus as mammalian cell expression vector for gene therapy: an emerging strategy. Mol Ther. 6, 5-11.
He, Y., Wei, M., Wu, Y., Qin, H., Li, W., Ma, X., Cheng, J., Ren, J., Shen, Y., Chen, Z., Sun, B., Huang, F. D., Shen, Y., and Zhou, Y. D. (2019). Amyloid β oligomers suppress excitatory transmitter release via presynaptic depletion of phosphatidylinositol-4,5-bisphosphate. Nat Commun. 10, 1193.
Kaufman, R. J. (2000). Overview of vector design for mammalian gene expression. Mol Biotechnol. 16, 151-60.
Kim, B. W., Choi, M., Kim, Y. S., Park, H., Lee, H. R., Yun, C. O., Kim, E. J., Choi, J. S., Kim, S., Rhim, H., et al. (2008). Vascular endothelial growth factor (VEGF) signaling regulates hippocampal neurons by elevation of intracellular calcium and activation of calcium/calmodulin protein kinase II and mammalian target of rapamycin. Cell Signal 20, 714-25.
Koffie, R. M., Meyer-Luehmann, M., Hashimoto, T., Adams, K. W., Mielke, M. L., Garcia-Alloza, M., Micheva, K. D., Smith, S. J., Kim, M. L., Lee, V. M., et al. (2009). Oligomeric amyloid beta associates with postsynaptic densities and correlates with excitatory synapse loss near senile plaques. Proc. Natl. Acad. Sci. USA 106, 4012-7.
Lacor, P. N., Buniel, M. C., Chang, L., Fernandez, S. J., Gong, Y., Viola, K. L., Lambert, M. P., Velasco, P. T., Bigio, E. H., Finch, C. E., et al. (2004). Synaptic targeting by Alzheimer's-related amyloid beta oligomers. J. Neurosci. 24, 10191-200.
Lacor, P. N., Buniel, M. C., Furlow, P. W., Clemente, A. S., Velasco, P. T., Wood, M., Viola, K. L., and Klein, W. L. (2007). Abeta oligomer-induced aberrations in synapse composition, shape, and density provide a molecular basis for loss of connectivity in Alzheimer's disease. J. Neurosci. 27, 796-807.
Lambert, M. P., Barlow, A. K., Chromy, B. A., Edwards, C., Freed, R., Liosatos, M., Morgan, T. E., Rozovsky, I., Trommer, B., Viola, K. L., et al. (1998). Diffusible, nonfibrillar ligands derived from Abetal-42 are potent central nervous system neurotoxins. Proc. Natl. Acad. Sci. USA 95, 6448-53.
Lesné, S., Koh, M. T., Kotilinek, L., Kayed, R., Glabe, C. G., Yang, A., Gallagher, M., and Ashe, K. H. (2006). A specific amyloid-beta protein assembly in the brain impairs memory. Nature 440, 352-7.
Li, S., Hong, S., Shepardson, N. E., Walsh, D. M., Shankar, G. M., and Selkoe, D. (2009). Soluble oligomers of amyloid Beta protein facilitate hippocampal long-term depression by disrupting neuronal glutamate uptake. Neuron. 62, 788-801.
Licht, T., Goshen, I., Avital, A., Kreisel, T., Zubedat, S., Eavri, R., Segal, M., Yirmiya, R., and Keshet, E. (2011). Reversible modulations of neuronal plasticity by VEGF. Proc Natl Acad Sci USA. 108, 5081-6.
Lue, L. F., Kuo, Y. M., Roher, A. E., Brachova, L., Shen, Y., Sue, L., Beach, T., Kurth, J. H., Rydel, R. E., and Rogers, J. (1999). Soluble amyloid beta peptide concentration as a predictor of synaptic change in Alzheimer's disease. Am. J. Pathol. 155, 853-62.
Martin L, Bouvet P, Chounlamountri N, Watrin C, Besançon R, Pinatel D, Meyronet D, Honnorat J, Buisson A, Salin P A, Meissirel C. VEGF counteracts amyloid-β-induced synaptic dysfunction. Cell Rep. 2021; 35:109121.
McLean, C. A., Cherny, R. A., Fraser, F. W., Fuller, S. J., Smith, M. J., Beyreuther, K., Bush, A. I., and Masters, C. L. (1999). Soluble pool of Abeta amyloid as a determinant of severity of neurodegeneration in Alzheimer's disease. Ann. Neurol. 46, 860-6.
Merrifield, B. (1986). Merrifield B. Solid phase synthesis. Science. 232,341-7.
Pickett, E. K., Koffie, R. M., Wegmann, S., Henstridge, C. M., Herrmann, A. G., Colom-Cadena, M., Lleo, A., Kay, K. R., Vaught, M., Soberman, R., Walsh, D. M., Hyman, B. T., and Spires-Jones, T. L. (2016). Non-Fibrillar Oligomeric Amyloid-β within Synapses. J Alzheimers Dis. 53, 787-800.
Renner, M., Lacor, P. N., Velasco, P. T., Xu, J., Contractor, A., Klein, W. L., and Triller, A. (2010). Deleterious effects of amyloid beta oligomers acting as an extracellular scaffold for mGluR5. Neuron 66, 739-54.
Shankar, G. M., Li, S., Mehta, T. H., Garcia-Munoz, A., Shepardson, N. E., Smith, I., Brett, F. M., Farrell, M. A., Rowan, M. J., Lemere, C. A., et al. (2008). Amyloid-beta protein dimers isolated directly from Alzheimer's brains impair synaptic plasticity and memory. Nat. Med. 14, 837-42.
Tam, J. P., Heath, W. F., and Merrifield R. B. (1983). SN 1 and SN 2 mechanisms for the deprotection of synthetic peptides by hydrogen fluoride. Studies to minimize the tyrosine alkylation side reaction. Int J Pept Protein Res. 21, 57-65.
Ting, J. T., Kelley, B. G., Lambert T. J., Cook, D. G., and Sullivan J M. (2007). Amyloid precursor protein overexpression depresses excitatory transmission through both presynaptic and postsynaptic mechanisms. Proc Natl Acad Sci USA. 104, 353-8.
Walsh, D. M., Klyubin, I., Fadeeva, J. V., Cullen, W. K., Anwyl, R., Wolfe, M. S., Rowan, M. J., and Selkoe, D. J. (2002). Naturally secreted oligomers of amyloid beta protein potently inhibit hippocampal long-term potentiation in vivo. Nature 416, 535-9.

1. A peptide comprising the amino acid sequence KRKKSRYKSWSVYVG (SEQ ID NO: 1).

2. A nucleic acid encoding the peptide of claim 1.

3. A method for treating a subject suffering from a neurodegenerative disorder comprising administering to said subject a therapeutically effective amount of an inhibitor of the interaction between amyloid-beta oligomers (Aβo) and vascular endothelial growth factor (VEGF).

4. The method according to claim 3 wherein the inhibitor of the interaction between (Aβo) and (VEGF) is a peptide.

5. The method according to claim 3 wherein the inhibitor is a peptide comprising or consisting of the amino acid sequence SEQ ID NO: 1.

6. The method according to claim 3 wherein said inhibitor i) targets Aβo and ii) inhibits Aβ aggregation.

7. The method according to claim 3 wherein the neurodegenerative disorder is selected from the group consisting of: Alzheimer disease (AD), Cerebral amyloid angiopathy (CAA), Down syndrome, Parkinson disease, Amyotrophic lateral sclerosis (ALS), and Motor neuron disease.

8. The method according to claim 3 further comprising administering to the subject a classical treatment of the neurodegenerative disorder, wherein the inhibitor and the classical treatment are administered as a combined preparation for simultaneous, separate or sequential administration.

9. The method according to claim 8, wherein the classical treatment is selected from the group consisting of: acetylcholinesterase inhibitor; N-methyl-D-aspartate (NMDA) receptor antagonist, PRX012, Aducanumab, and Masitinib.

10. A pharmaceutical composition comprising an inhibitor of the interaction between amyloid-beta oligomers (Aβo) and the vascular endothelial growth factor (VEGF).

11. The pharmaceutical composition according to claim 10, wherein the inhibitor is a peptide comprising or consisting of the amino acid sequence SEQ ID NO: 1.

12. The pharmaceutical composition according to claim 11 further comprising at least one pharmaceutically acceptable excipients, and optionally a sustained-release matrix.

13. (canceled)

14. The pharmaceutical composition according to claim 12 wherein the sustained-release matrix comprises biodegradable polymers and/or nanoparticles.
